(19) United States
(12) Patent Application Publication
(10) Pub. No.: US 2013/0142779 A1
(43) Pub. Date: Jun. 6, 2013

(54) Treatment of Hi-Virus infections with oxidised blood proteins

(71) Applicant: Hamburger Stiflung Zur Foerderung Von Wissenschaft Und Kultur, Hamburg (DE)

(72) Inventor: Beate Kehrel, Muenster (DE)

(73) Assignee: Hamburger Stiftung Zur Foerderung Von Wissenschaft Und Kultur, Hamburg (DE)

(21) Appl. No.: 13/650,305

(22) Filed: Oct. 12, 2012

Related U.S. Application Data
(63) Continuation of application No. 10/555,423, filed on Oct. 25, 2005, now abandoned.

(51) Int. Cl. C07K 14/765

(52) U.S. Cl. CPC C07K 14/765
USPC 424/130.1; 514/3.8

(57) ABSTRACT

The invention relates to the field of medicaments for combating an infection of a host cell by HI viruses and/or for inhibiting binding of an Env protein to a CD4 protein. For these purposes, the invention provides medicaments which comprise oxidized proteins, oxidized peptides and/or peptidomimetics of such oxidized proteins and/or oxidized peptides, as well as preparation processes for such medicaments and therapeutic and non-therapeutic possible uses of these medicaments.

### CROSS REFERENCE TO RELATED APPLICATION

This application is a Continuation application of U.S. patent application Ser. No. 10/555,423 which is a national stage of PCT/EP2003/004374 filed Apr. 25, 2003, the entire contents of which are incorporated herein by reference.

### BACKGROUND OF THE INVENTION

1. Field of the Invention

The invention relates to the field of medicaments for combating an infection of a host cell by HI viruses and/or for inhibiting binding of an Env protein to a CD4 protein. For these purposes, the invention provides medicaments which comprise oxidized proteins, oxidized peptides and/or peptidomimetics of such oxidized proteins and/or oxidized peptides, as well as preparation processes for such medicaments and therapeutic and non-therapeutic possible uses of these medicaments. Oxidized proteins, oxidized peptides and the abovementioned peptidomimetics are called collectively by the term “oxP” in the following.

2. Related Art of the Invention

As described in WO 02/22150 A2 and WO 02/32445 A2, oxP, such as, but not limited to, “immune defence activated” antithrombin (IDA-ATIII), oxidized serum albumin, oxidized fibrinogen, bind to the GP120 of the Env protein in the envelope of the HI virus. As demonstrated by us in WO 02/22150 A2 by way of example for IDA-ATIII, oxPs are capable of preventing the multiplication of the HI virus in the host cell.

### SUMMARY OF THE INVENTION

In the context of the present invention, we demonstrate that oxPs are capable of preventing contact between the HI virus and the host cell, of blocking the formation of syncytia from infected and non-infected defence cells and therefore of already inhibiting the infection at the “entry” level. Surprisingly, it has furthermore been found that no purified oxidized proteins and oxidized peptides, such as human serum albumin, are necessary to suppress infection of a host cell with HI viruses, but that oxidized blood plasma as such can already prevent an infection at the “entry” level.

Although some potent antiviral medicaments exist, HIV has developed into a true worldwide pandemic and has therefore become the most important infectious disease. Over 4 million people currently die from this disease every year. Since HIV medicaments which are approved today, such as protease inhibitors and reverse transcriptase inhibitors, are not capable of removing HIV completely from infected persons, there is the urgent need to discover novel antiviral medicaments.

HIV infections/AIDS represent one of the most important crises in the development of humanity. An HIV medicament must therefore be found which, in addition to meeting all the scientific requirements, on the one hand is easy to prepare and is attainable, and on the other hand can be made readily accessible to people specifically in the sub-Sahara regions.

These problems have been solved with the present invention, since the oxPs provided here already block HIV infections at the very first entry level, oxPs are easy and cheap to prepare (protein from the plasma of a human can be used directly) and this process can be carried out not only in special laboratories.

Enveloped viruses in principle penetrate into their target cell in that the membrane of the virus comes into contact with the membrane of the target cell and the two membranes then become so close to one another that they fuse. For contact of the HIV-1 virus with the host cell, binding of the external HIV envelope protein Env to the CD4 receptor is necessary. This takes place via the Env constituent GP120. GP120 subsequently binds to one of the main co-receptors CXCR4 or CCR5. The binding of GP120 to its receptors leads to a change in the conformation of the external GP120/GP41 complex. This interaction is a basic prerequisite for rendering possible the provision of the viral GP41 N terminus, which in the end leads to the membrane fusion.

Neutralizing antibodies which are directed directly against GP120 are one factor which can partly block the viral entry. However, neutralizing antibodies of natural infections have only a limited efficiency, since on the one hand HIV produces a very large number of different antigen variants, and on the other hand after an infection a restricting “clonal dominance” of HIV-neutralizing antibodies occurs. New HIV-1 variants can therefore easily escape such a highly specific but restricted response.

The most recent findings have shown that the non-specific inherited immune response is relevant for defence against HIV. Polymorphonuclear neutrophilic leukocytes (PMNL) and monocytes are viricidal towards HIV-1 after stimulation. Lipopolysaccharide (LPS) stimulation, which leads to oxidative bursting of leukocytes, causes a blockade of the HIV entry, without taking into consideration the viral co-receptor phenotype.

Leukocytes generate H₂O₂ and secrete the haem protein myeloperoxidase (MPO). Klebanoff and his colleagues have shown that stimulated PMNL from patients with inherited deficiency of the enzyme MPO had a reduced viricidal activity. By addition of MPO, it was possible to reconstitute the reduced defence power again. On the basis of the current state of knowledge, it is assumed that the MPO product HOCl itself is the agent having an antiviral action. The expression and the release of MPO, which produces HOCl, is strictly controlled in vivo. Free HOCl represents a part of oxidative stress.

### BRIEF DESCRIPTION OF THE DRAWINGS

FIG. 1: IDA-HSA is not cytotoxic. Hela cells which express human CD4, CXCR4 and CCR5 were cultured in the presence of [³H]-tymidine and various concentrations of IDA-HSA (□) or HSA (▴). The cells were sown at 10⁴ cells per “well” in a 96 “well” plate in triplicate. On day 2, [³H]-tymidine (10 μCi/ml) was added to each “well”. After 8 hours, the DNA was harvested and bound to a glass fibre membrane and the [³H]-tymidine incorporated was quantified with a β-counter.

FIG. 2: IDA-HSA binds to HIV-1 GP120. The specific binding of IDA-HSA to GP120 was illustrated in a standard ELISA method (a) and by surface plasmon resonance spectroscopy (SPR) (b). For the ELISA, a 96-well plate was coated with 100 μl/well of recombinant GP120 (1 μg/ml) and then blocked with 0.25% gelatine (in PBS) (1 h RT). IDA-HSA (∘) and HSA () were applied in various concentrations (0, 0.25, 0.5, 1, 2, 5, 10, 20 μg/ml in PBS). Bound protein was detected with HRP-conjugated, polyclonal, specific anti-HSA antibodies (Sigma). For the SPR, GP120 (10 μg/ml) was bonded covalently with EDC/NHC on a dextran-coated, CH-activated sensor chip (CMS, Biacore, Sweden). The flow rate was 5 μl/min for 10 min. After blocking with ethanolamine, the binding of IDA-HSA and HSA (in each case 1 μg/ml) was analysed at a flow rate of 5 μl/min for 6 minutes.

FIG. 3: Oxidized plasma protein mixtures bind to HIV-GP120. The specific binding of a mixture of oxidized plasma proteins to GP120 was demonstrated by surface plasmon resonance spectroscopy (SPR). GP120 (10 μg/ml) was bonded covalently on a sensor chip (C1, Biacore, Sweden).

| | |
|---|---|
| Injection of 20 μl 100 mM glycine, | Purification of the sensor |
| 0.3% Triton X-100 pH 12 (twice) | surface |
| 5 μl/min | Flow rate |
| Injection of 20 μl 400 mM EDTA | Removal of calcium ions |
| | from the sensor surface |
| Injection of 50 μl NHS/EDC | Activation of the sensor surface |
| Injection of 20 μl 100 nM P120 in | Covalent coupling of P120 |
| 10 mM NaAc pH 4 (dilution 1:10) | |
| Injection of 55 μl ethanolamine | Blocking of the remaining |
| | activated esters |

A solution of 93.5 nM PluO was then injected via immobilized P120. An unambiguous binding curve (see above) was obtained. In subsequent experiments, it was shown that the resulting signal height correlates with the amount of immobilized P120.

FIG. 4: Inhibition of the HIV replication. GHOST-CXCR4 or GHOST-CCR5 cells were infected with X4-tropic NL4-3 (triangles) or R5-tropic NL-991 viruses (squares) (500TCID₅₀). On day 5, the cell culture supernatant was tested for p24 antigen with a p24 standard ELISA. The mean of 3 measurements is shown. The standard error for the mean was <10%, NL4-3+IDA-HSA (▴); NL4-3+HSA (Δ); NL-991+IDA-HSA (▪); NL-991+HSA (□).

FIG. 5: Inhibition of the HIV-induced syncytia formation. GHOST-CXCR4 or GHOST-CCR5 cells were infected with 500 TCID₅₀ of the HIV laboratory isolates (A-E) NL4-3 (X4-monotropic) or (F-L) NL-991 (R5-monotropic). The infection was inhibited by addition of IDA-HSA protein to the culture medium with a final concentration of 0, 2, 5, 10 or 20 μg/ml. 5 days after the start of infection, the infection was rendered visible by demonstration of the syncytia induced and of the destruction of the cell lawn. For this, the cells/nuclei were stained by a standard eosin/methylene blue/azure staining procedure (Hemacolor, Merck). (a), NL4-3 infected cells; (b) NL4-3+2 μg/ml IDA-HSA; (c) NL4-3+5 μg/ml IDA-HSA; (d) NL4-3+10 μg/ml IDA-HSA; (e) NL4-3+20 μg/ml IDA-HSA; (g) NL-991 infected cells; (h) NL-991+2 μg/ml IDA-HSA; (i) NL-911+5 μg/ml IDA-HSA; (j) NL-911+10 μg/ml IDA-HSA; (k) NL-911+20 μg/ml IDA-HSA; (l) 20 μg/ml IDA-HSA.

FIG. 6: Inhibition of the GP160-induced syncytia formation. Hela cells which express human CD4, CXCR4 and CCR5 were transfected with pSVATGrev plasmids, which express NL4-3 or NL-911 env. For this, either IDA-HSA or HSA protein was added (final concentration 20 and 50 μg/ml). The syncytia formation was investigated after 28 hours by standard phase contrast microscopy of the living cells. IDA-HSA prevented syncytia formation in a dose-dependent manner.

### DETAILED DESCRIPTION OF THE INVENTION

In the context of the invention, we have now found that protein/peptides which has/have come into contact with HOCl can be transformed into an antiviral form, and as a result HOCl has an indirect action against HIV-1, in addition to the known direct action.

Relevance of this type of inhibition for future therapeutic applications: HOCl, which is produced by MPO in inflamed tissue, modifies LDL and many other human proteins in vivo. HOCl-modified proteins may be detected by specific monoclonal antibodies (WO-02/32445 A2). A structural change generated by HOCl treatment therefore provides an epitope which is already present in vivo and therefore well-known to the immune system. OxPs should therefore be tolerated in vivo.

In addition, oxP is based on a relatively simple chemical modification associated with low costs.

Why does the body not produce enough oxP in vivo to protect itself from HIV infection? One answer to this question could be the observation that the function of the neutrophilic leukocytes and of the monocytes deteriorates in HIV-infected persons at the start of the infection, and that the losses of function correlate positively with the extent/progression of the HIV-induced disease.

The object of the present invention was therefore to provide a medicament which already inhibits an HIV infection at the “entry” level, and at the same time is easy and inexpensive to prepare and therefore can also be used for patients in the so-called “third world”.

The object is achieved by a preparation process for a medicament for combating an infection of a host cell by HI viruses and/or for inhibiting binding of an Env protein to a CD4 protein, which is characterized by the steps:
a) provision of a mixture, comprising proteins and/or peptides, which can be obtained from whole human and/or animal blood
b) oxidation of the proteins and/or peptides contained in the mixture.

While in earlier studies the action of individual, purified proteins on the binding of an Env protein to a CD4 protein was demonstrated, it has now emerged, surprisingly, that the infection of a host cell by HI viruses and/or the binding of an Env protein to a CD4 protein can also be inhibited, reduced or even completely suppressed by protein mixtures. In particular, these actions can also be achieved by complex protein- and/or peptide-containing mixtures having more than one variety of protein or peptide. This was surprising, since it was to be expected that the process for the preparation of the oxidized proteins and/or peptides necessary for combating an infection of a host cell by HI viruses and/or for inhibiting the binding of an Env protein to a CD4 protein is impaired during the oxidation in such complex mixtures by achieving false conformations and by side reactions such that the activity of oxidized proteins in such mixtures is reduced or eliminated completely.

In the context of this invention, whole blood is the blood taken from a human or animal, in particular a monkey, cow, sheep, pig, dog, goat, rabbit or mouse, and optionally mixed with a suitable anticoagulant.

In the context of the present invention, as already stated above, it has been found that oxP blocks the binding of HIV-GP120 to CD4 and thus prevents the penetration of the HIV into the target cell. Since a virus is dependent upon penetration into a host cell for its multiplication, however, it cannot multiply further. The formation of syncytia from HIV-infected defence cells with non-infected defence cells does not take place, so that these pursue their task further and can destroy infected cells.

It has proved to be particularly advantageous if the mixture oxidized in step b) comprises a plasma protein and/or a plasma peptide which is or are oxidized in step b). Plasma proteins and plasma peptides here are those proteins and peptides which are contained in the liquid which settles on the top on centrifugation or sedimentation of whole blood, and the proteins, peptides and protein or peptide complexes which can be prepared from them. The plasma proteins and plasma peptides include, in particular, serum albumin, in particular human serum albumin and bovine serum albumin, antithrombin, “immune defence activated” antithrombin (IDA-ATIII), fibrinogen, coagulation factors and immunoglobulins. It is therefore furthermore preferable to use a plasma protein, plasma peptide, serum albumin, in particular human serum albumin and bovine serum albumin, antithrombin, “immune defence activated” antithrombin (IDA-ATIII), fibrinogen, a coagulation factor or an immunoglobulin or mixtures of these substances for the preparation of a medicament for combating an infection of a host cell by HI viruses and/or for inhibiting binding of an Env protein to a CD4 protein.

Instead of or in addition to oxidized proteins and/or oxidized peptides, the medicament can also be prepared by mixing of a pharmaceutically acceptable carrier with a peptidomimetic, which can replace an oxidized protein and/or an oxidized peptide of a mixture oxidized in step b) by one of the processes according to the invention which are described above. Research into pharmaceutical chemistry, in particular, has proposed strategies for the preparation of peptidomimetics (cf. Rompp online, document identification RD-16-00950). By using these strategies, it is possible for the person skilled in the art to produce the peptidomimetics necessary for carrying out the preparation process according to the invention.

The medicament prepared by the process thus comprises oxidized protein(s), oxidized peptide(s), oxidized amino acids, peptidomimetics and/or peptide analogues (summarized as oxP).

In the context of the present invention, the medicament can comprise either the complete oxP, which can be prepared, for example, by the process described in the examples. However, oxP formed after defence reactions can also be isolated from the body. Furthermore, plasma fractions (plasma protein mixtures) of the patient himself or of a donor, without necessary isolation of the proteins, can be converted directly into a medicament which comprises oxP. It is furthermore conceivable to use ox-peptides which bind to HIV-GP120. Analogues of oxP are also suitable in the context of the present invention if they likewise prevent the entry of HIV into its target cells.

A medicament according to the invention can of course comprise further pharmaceutically acceptable auxiliary or/and carrier substances, the medicament being formulated for local, intradermal, superficial, intraperitonal, intravenous, intramuscular or oral administration or rendering possible its administration via vesicles. The medicament according to the invention therefore preferably comprises those auxiliary and carrier substances which render possible the particular preferred mode of administration.

The medicament according to the invention can of course comprise, in addition to oxP, parts or analogues or mimetics thereof, further substances, such as, for example, antibiotics, other HIV infection inhibitors, etc. Depending on the concomitant disease to be treated, it may be of advantage to provide supporting treatment with known medicaments. An appropriate combination of this medicament with oxP is therefore optionally a preferred embodiment of the present invention.

Advantageous uses of the medicaments according to the invention are described in the claims. A particularly advantageous non-therapeutic use comprises the treatment of cells and/or cell cultures, in each case in particular of animal or human origin, for combating an infection of a host cell by HI viruses, in particular by inhibition of the binding of an Env protein to a CD4 protein, and in this context in particular for inhibiting the binding between a GP120 unit of an Env protein to a CD4 protein. The medicaments according to the invention can furthermore be used for binding and optionally for detection of an Env protein, in particular a GP120 unit of an Env protein. The invention is described in more detail in the following with the aid of the examples and the figures.

1.) Example for the preparation of oxP (see also our Application WO 02/32445 A2)

In order to transform normal human serum albumin into the antiviral form, HSA was activated with HOCl. Freshly prepared HOCl was added to HSA in a molar ratio of 1:100. After an incubation time of 30 minutes at room temperature, the hypochlorite which remained was removed by gel filtration (Sephadex G25).

In another use, protein mixtures were isolated from human plasma by a standard method (e.g. ammonium sulfate precipitation/desalination or cryoprecipitation) and these were then modified directly with freshly prepared HOCl, as described for human serum albumin.

2.) OxP is not cytotoxic.

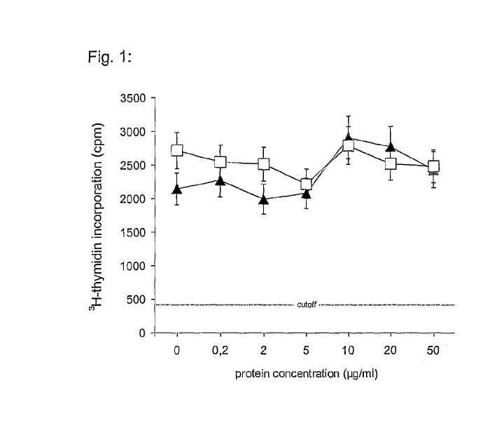

The HOCl-modified HSA was first tested in the ³H-tymidine incorporation assay. Up to a use concentration of 50 μg/ml, no anticellular activity in respect of the cell proliferation of Hela or GHOST cells compared with normal HSA was to be observed (FIG. 1).

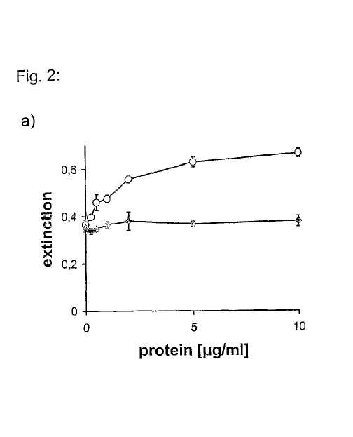

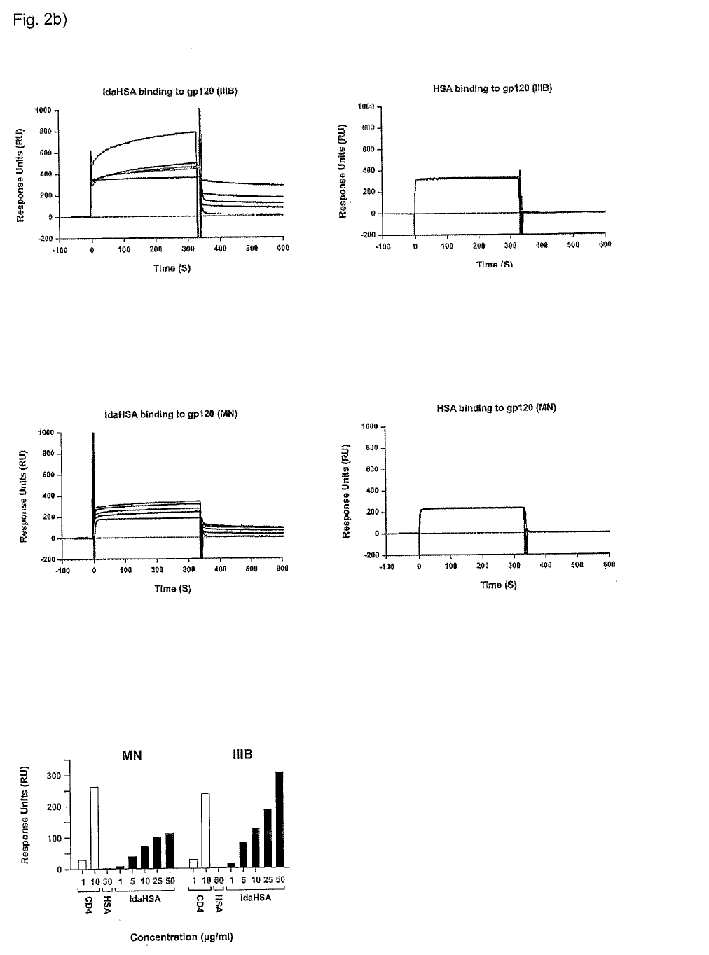

3.) The binding of oxP to IIIB GP120 (from the AIDS reagent EVA project) was illustrated in a standard ELISA assay (FIG. 2a). In addition, the binding of oxP both to IIIB GP120 and to SF2 GP120 was demonstrated in surface plasmon resonance spectroscopy (SPR) (FIG. 2b). In both experiments, the direct interaction of oxP on GP120 was demonstrated. Only after transformation of the protein into the oxP form was a specific binding to be observed. If normal protein, in this case HSA, normal bovine serum albumin, glutathione S-transferase (GST) or fusion protein with a GST-VS, which included the FP120 V3 loop, was used, no binding was to be observed. In all these control experiments, the “response units” (RU) were <5. In addition to the SPR binding study, the kinetics of the oxP (here ox ATIII) GP120-IIIB interaction were investigated. The analysis gave ka and kd values of 1.47*10⁻⁹ and 7.01*10⁻¹⁰ M, and resulting from these a KD of 7.0*10⁻¹⁰ M (Rmax=120; Chi2=40).

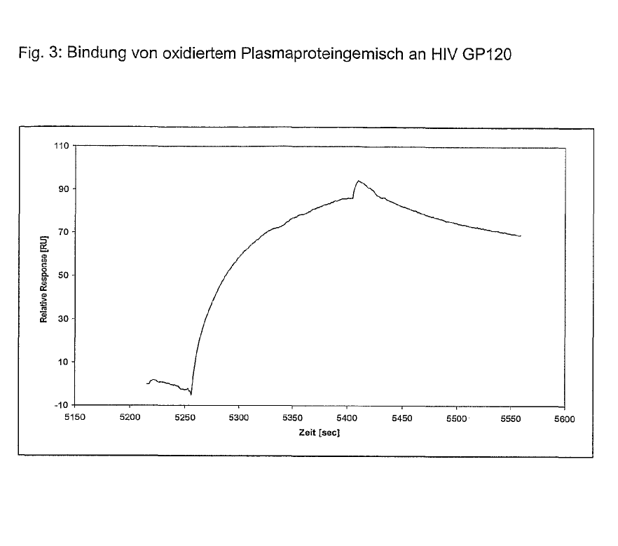

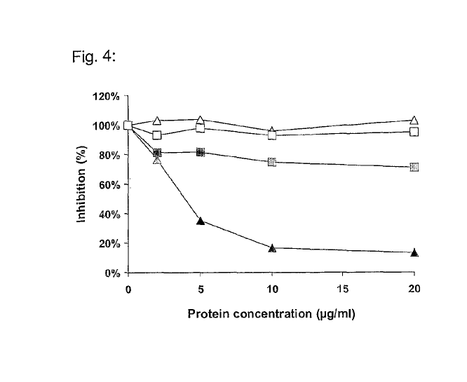

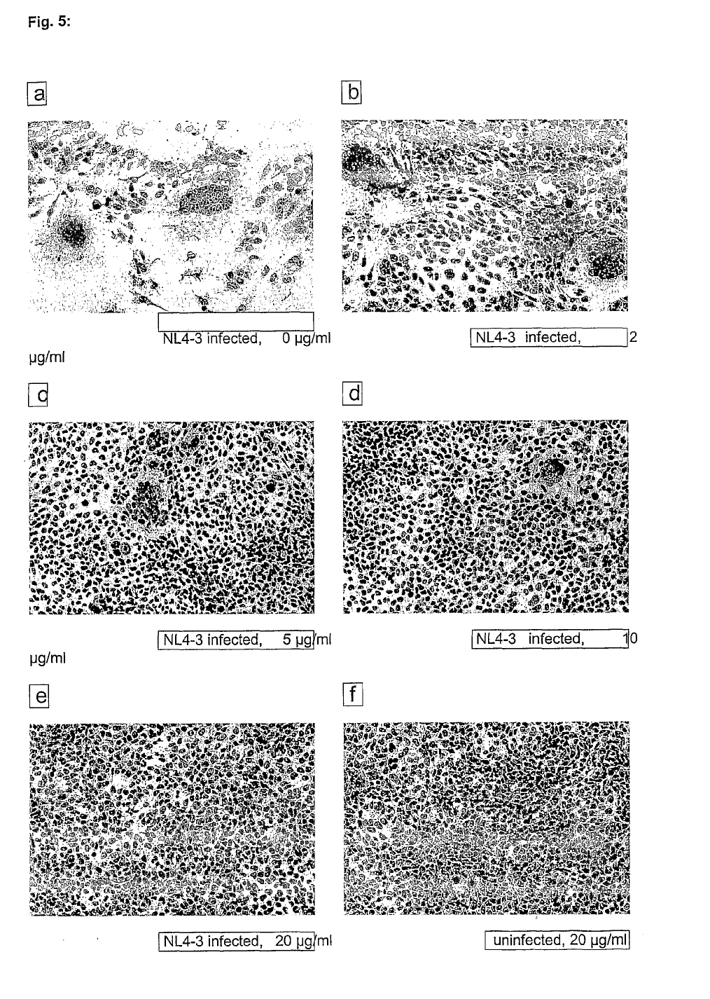

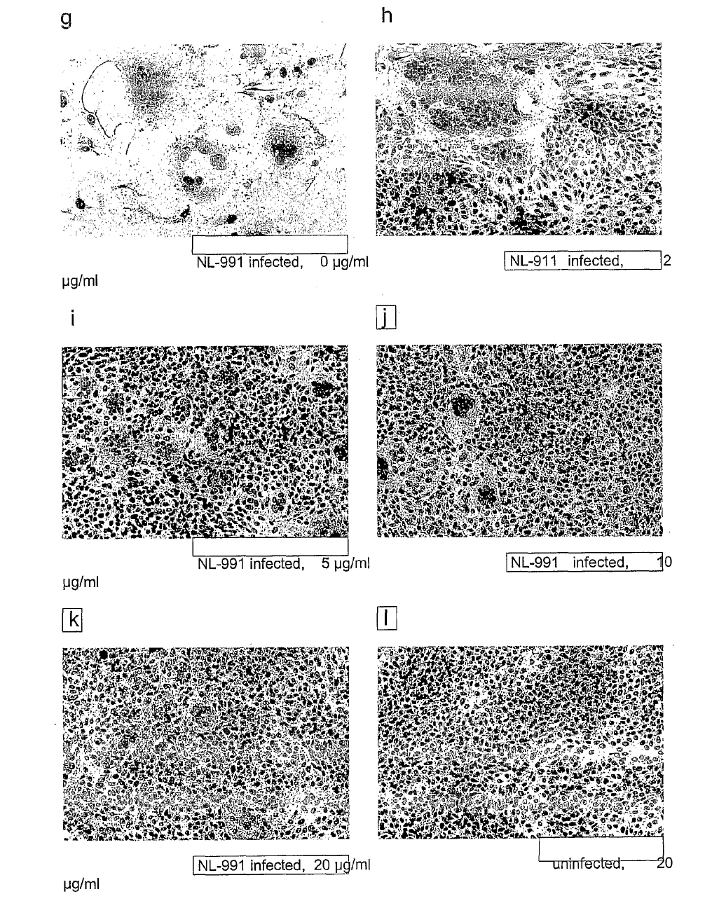

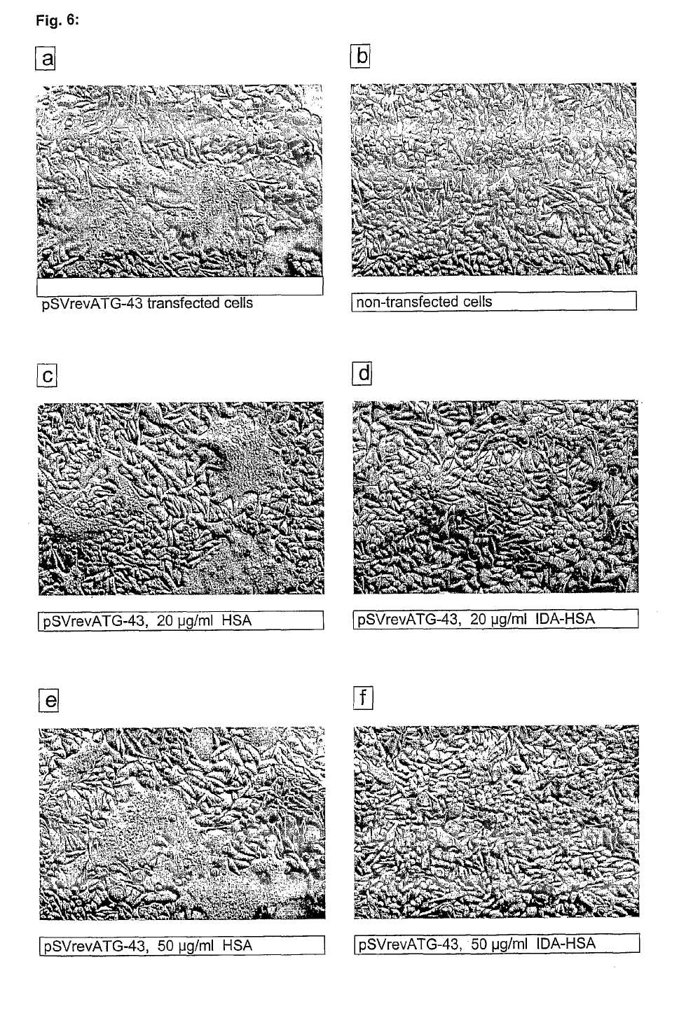

4.) A non-fractionated protein mixture from human plasma binds to HIV-GP120 after treatment with HOCl, as described above. (FIG. 3)

5.) OxP neutralizes HIV

For HIV neutralization experiments, the HIV-1 strains NL4-3 and a variant of the NL4-3, NL-991, in which the V3 loop was exchanged for a V3 loop of the primary isolate PI-991, were used. NL4-3 is a monotropic virus which uses only the CXCR4 co-receptor. The NL-991 virus is R5-monotropic and uses only CCR5 as a co-receptor. FIG. 4 shows that the replication of both viruses is inhibited by oxP. This is reflected in the amount of HIV p24 antigen produced.

6.) In HIV cell cultures, HIV-infected cells fuse with non-infected CD4⁺ target cells. This fusion among cells is known as syncytia formation. This syncytia formation is to be attributed to the binding of GP120, which is expressed on the membrane of infected cells, to the CD4 receptor on the target cell and subsequent insertion of the GP41 N terminus into the target membrane. Both viruses which were used in this neutralization assay (NL4-3 and NL-991) were capable of forming syncytia with the GHOST-CXCR4 and GHOST-CCR5 cells (FIG. 5). It was possible for both the syncytia formation induced by the NL4-3 virus (FIG. 5a), and that induced by the NL-991 virus (FIG. 5g), to be inhibited by addition of oxP (in this case ox-HSA) in concentrations of up to 20 μg/ml (FIG. 5e-4k). oxP showed a dose-dependent inhibition (5b-e; 5h-k). As already shown in FIG. 1, oxP itself influenced neither cell proliferation nor cell morphology (FIG. 5f; 5I), and the staining of the cell nuclei proved the vitality of the GHOST cells.

7.) OxP blocks HIV infection at the “entry level”.

Syncytia formation is based on the presence of the viral envelope and the viral docking proteins on the membrane surface of the host cells. Hela-P4 cells (CD4+, CXCR4+, CCR5+), which additionally expressed the viral receptors GP120/GP41, were therefore used. For this, Hela-P4 cells were transfected with GP160 vectors, so that they expressed the Env proteins of HIV-NL4-3 and HIV-NL-911. Gp160-transfected Hela-P4 cells fused and, after 28 h, in contrast to non-transfected cells (6b; 6h), formed syncytia (FIG. 6a; 6g).

Incubation with oxP led to a dose-dependent inhibition of the formation of syncytia (6d, f, 6j, l), in contrast to incubation with non-modified protein (in this case, as an example, HSA) (6c, e, i, k). This transfection assay imitates the entry of X4- and R5-tropic HI viruses (NL4-3, NL-911). In both syncytia test methods, oxP showed “anti-HIV entry” activity at 20 and 50 μg/ml. This illustrates that oxP acts at the GP120-CD4 interaction level with an ID>95 at 50 μg/ml.

8.) Precipitation of plasma proteins and oxidation thereof:
10 ml citrate blood were centrifuged at 3,200 rpm/2,000 g for 10 minutes and the supernatant plasma was removed. 5 M (NH4)2SO4 was added in a volume ratio of 1:1, the plasma was stirred for 20 minutes and the plasma proteins were thereby precipitated. The suspension was centrifuged again for 10 minutes at 3,200 rpm/2,000 g, the supernatant was discarded and the precipitated proteins were resuspended in PBS buffer (pH 7.4). The solution was introduced into a dialysis hose (exclusion limit 10,000 D) and dialysed against PBS buffer for 3 days. During this procedure, the buffer was changed 3 times. Alternatively, the protein mixture was desalinated by a gel filtration process.

After the dialysis/gel filtration, the total protein content was determined photometrically by standard methods.

Oxidation to the medicament according to the invention: Fresh HOCl (12 μl) was added to 500 μg of the protein solution and the mixture was topped up to a volume of 1 ml with PBS/0.1 mM EDTA buffer. After a reaction time of 15 minutes on ice (0° C.), the solution was introduced onto a gel filtation column equilibrated with PBS buffer and the unreacted HOCl was removed in this way.

### REFERENCES

Stephenson J: Growing, Evolving HIV/AIDS Pandemic Is Producing Social and Economic Fallout

JAMA. 2003; 289:31-33.

2 Brenner B G, Turner D, Wainberg M A: HIV-1 drug resistance: can we overcome?

Expert Opin Biol Ther. 2002; 2(7):751-61.

3 Kwong P D, Wyatt R, Robinson J, Sweet R W, Sodroski J, Hendrickson W A. Structure of an HIV gp120 envelope glycoprotein in complex with the CD4 receptor and a neutralizing human antibody. Nature. 1998; 393(6686):648-59

4 Gaschen B, Taylor J, Yusim K, Foley B, Gao F, Lang D, Novitsky V, Haynes B, Hahn B H, Bhattacharya T, Korber B.
Diversity considerations in HIV-1 vaccine selection.

Science. 2002; 296(5577):2354-60.

5 Muller S, Wang H, Silverman G J, Bramlet G, Haigwood N, Kohler H. B-cell abnormalities in AIDS: stable and clonally-restricted antibody response in HIV-1 infection.

Scand J Immunol. 1993; 38(4):327-34.

6 Nara P L, Garrity R R, Goudsmit J.
Neutralization of HIV-1: a paradox of humoral proportions.

FASEB J. 1991 5(10):2437-55.

7 Verani A, Sironi F, Siccardi A G, Lusso P, Vercelli D.
Inhibition of CXCR4-tropic HIV-1 infection by lipopolysaccharide: evidence of different mechanisms in macrophages and T lymphocytes.

J Immunol. 2002; 168(12):6388-95.

8 Chase M J, Klebanoff S J
Viricidal effect of stimulated human mononuclear phagocytes on human immunodeficiency virus type 1.

Proc Natl Acad Sci USA. 1992, 15; 89(12):5582-5.

9 Klebanoff S J, Coombs R W.
Viricidal effect of polymorphonuclear leukocytes on human immunodeficiency virus-1. Role of the myeloperoxidase system.

J Clin Invest. 1992; 89(6):2014-7.

10 Polzer S, Dittmar M T, Schmitz H, Schreiber M.
The N-linked glycan g15 within the V3 loop of the HIV-1 external glycoprotein gp120 affects coreceptor usage, cellular tropism, and neutralization.

Virology. 2002, 5; 304(1):70-80.

11 Daugherty A, Dunn J L, Rateri D L, Heinecke J W.
Myeloperoxidase, a catalyst for lipoprotein oxidation, is expressed in human atherosclerotic lesions. J Clin Invest. 1994; 94(1):437-44.

12 Sugiyama S, Okada Y, Sukhova G K, Virmani R, Heinecke J W, Libby P.
Macrophage myeloperoxidase regulation by granulocyte macrophage colony-stimulating factor in human atherosclerosis and implications in acute coronary syndromes.

Am J Pathol. 2001; 158(3):879-91

13 Spada C, Treitinger A, Fujimura A Y.
Morphofunctional Study of Blood Polymorphonuclear Leucocytes in HIV-Seropositive Individuals.

Braz J Infect Dis. 1998; 2(6):285-290

14 Karen C. Hayani, Stephen C. Venal, and David L. Pitrak
Impaired Phagocyte Oxidative Capacity in Human Immunodeficiency Virus Infected

The Journal of Infectious Diseases 1999; 179:584-589

15 WO-A-02/22150

16 WO-A-02/32445

1-9. (canceled)

10. A method for combating an infection of a host cell by HI viruses, comprising administering to a subject in need thereof a medicament prepared by the steps: a) providing a mixture comprising human and/or animal blood plasma, and b) oxidating the proteins and/or peptides of the blood plasma which are contained in the mixture with HOCl.

11. The method of claim 10, wherein one of the proteins contained in the mixture and oxidized in step b) is serum albumin, antithrombin, immune defence activated antithrombin (IDA-ATIII), fibrinogen, coagulation factor or an immunoglobulin.

12. The method of claim 11, wherein said serum albumin is human serum albumin or bovine serum albumin.

13. A method for inhibition of binding of an Env protein to a CD4 protein, comprising administering to a subject in need thereof a medicament prepared by the steps: a) providing a mixture comprising human and/or animal blood plasma, and b) oxidating the proteins and/or peptides of the blood plasma which are contained in the mixture with HOCl.
